(19) United States
(12) Patent Application Publication
(10) Pub. No.: US 2015/0374751 A1
(43) Pub. Date: Dec. 31, 2015

(54) Implantable, Malleable Calcium Phosphate Compositions and Methods for Making and Using the Same

(71) Applicant: Skeletal Kinetics, LLC, Cupertino, CA (US)

(72) Inventors: David C. Delaney, Capitola, CA (US); Sahil Jalota, San Diego, CA (US); Duran N. Yetkinler, San Jose, CA (US)

(21) Appl. No.: 14/742,532

(22) Filed: Jun. 17, 2015

Related U.S. Application Data
(60) Provisional application No. 62/017,107, filed on Jun. 25, 2014.

(51) Int. Cl. A61K 33/42; A61L 27/12

(52) U.S. Cl. CPC A61K 33/42; A61L 27/12; A61L 2430/02

(57) ABSTRACT

Implantable, malleable calcium phosphate compositions are provided. The compositions include calcium and phosphate reactants and a non-aqueous liquid carrier. Aspects of the invention also include methods of making and using the compositions, as well as kits that include the compositions. The compositions and methods find use in a variety of applications, including the repair of hard tissue defects, e.g., bone defects.

### CROSS REFERENCE TO RELATED APPLICATIONS

Pursuant to 35 U.S.C. §119(e), this application claims priority to the filing date of U.S. Provisional Patent Application Ser. No. 62/017,107, filed on Jun. 25, 2014, the disclosure of which application is herein incorporated by reference in its entirety.

### INTRODUCTION

Calcium phosphate cements find use in a variety of different applications in the orthopedic and dental fields. Calcium phosphate cements can be used, for example, as a filler for bone voids in the treatment of hard tissue defects (e.g., fractures and osteoporotic bone), in the production of prosthetic orthopedic and dental implants, and as a vehicle for drug delivery. Materials that set into solid calcium phosphate mineral products are of particular interest as such products can closely resemble the mineral phase of natural bone and are susceptible to remodeling, making such products extremely attractive for use in orthopedics and related fields.

Calcium phosphate cements capable of setting into solid calcium phosphate products are typically prepared by combining a dry component(s) and a liquid to form a paste-like material. See, e.g., U.S. Pat. Nos. 6,719,993; 6,375,935; 6,027,742; 6,005,162; 5,997,624 and 5,976,234. Calcium phosphate cements prepared in such a manner begin to set upon mixing of the dry and liquid components. In some instances, for example, the calcium phosphate cements in less than 20 minutes. See e.g., U.S. Pat. No. 6,375,935. As such, mixing of the dry and liquid components is performed close to the time of usage. Moreover, once formed, the composition must be applied to the desired site and molded to the desired shape prior to setting into product.

### SUMMARY

Implantable, malleable calcium phosphate compositions are provided. The compositions include calcium and phosphate reactants and a non-aqueous liquid carrier. Aspects of the invention also include methods of making and using the compositions, as well as kits that include the compositions. The compositions and methods find use in a variety of applications, including the repair of hard tissue defects, e.g., bone defects.

### BRIEF DESCRIPTION OF THE FIGURES

FIG. 1 displays the FTIR pattern of a composition according to an embodiment of the invention following in vitro incubation for 72 hours, curing and drying.

FIG. 2 displays the XRD pattern of a composition according to an embodiment of the invention following in vitro incubation for 72 hours, curing and drying.

FIG. 3 shows x-ray diffraction patterns of a composition according to an embodiment of the invention following in vitro incubation for 72 hours.

FIG. 4 provides an SEM micrograph of a cured and dried product produced from a composition according to an embodiment of the invention.

FIG. 5: pH change versus Time for PBS with a composition of the present invention, Callos® Calcium phosphate cement (Skeletal Kinetics, Cupertino Calif.) and control (no cement).

FIG. 6 provides the setting time of a composition according to an embodiment of the invention, where specimens were inserted in 37° C. PBS solution immediately after mixed.

FIG. 7A provides the setting reaction temperature change for a composition according to an embodiment of the present invention and FIG. 7B provides the setting reaction temperature for Callos® calcium phosphate cement after immersion in PBS maintained at 37° C. for up to 24 hours. PBS served as control.

FIG. 8 provides the FTIR patterns for Callos® and a composition according to an embodiment of the invention.

FIG. 9 provides X-ray Diffraction patterns for Callos® and a composition according to an embodiment of the invention.

FIG. 10A provides an SEM micrograph for a cured composition according to an embodiment of the present invention and FIG. 10B provides an SEM micrograph for a cured composition produced from Callos® calcium phosphate cement.

### DETAILED DESCRIPTION

Implantable, malleable calcium phosphate compositions are provided. The compositions include calcium and phosphate reactants and a non-aqueous liquid carrier. Aspects of the invention also include methods of making and using the compositions, as well as kits that include the compositions. The compositions and methods find use in a variety of applications, including the repair of hard tissue defects, e.g., bone defects.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Compositions

In one aspect, provided herein are implantable, malleable calcium phosphate compositions. Such compositions include calcium and phosphate reactants and a non-aqueous liquid carrier. In the presence of an aqueous medium (e.g., a fluid in vivo environment), the calcium and phosphate reactants react to produce a product containing a calcium phosphate mineral that is different from the that of calcium and phosphate reactants, i.e., any calcium phosphate mineral contained in the product is different that the initial calcium and phosphate reactants.

As the compositions are malleable, they can be manipulated by hand into desired shapes, and can be finger packed in to a bone void defect. The compositions in some instances are pastes or putties, and may have a viscosity ranging in some instances from 150K to 100,000K centipoise, such as 500K to 50,000K centipoise.

As summarized above, the compositions include a calcium and phosphate reactant component. In certain embodiments, the calcium and phosphate reactants are particulate compositions, e.g., powders, where the particle size of the components of the particulate compositions typically ranges from 1 to 1000 microns, including from 1 to 200 microns and from 1 to 40 microns.

The calcium and phosphate reactants of the subject compositions may be present as two or more different compounds. In some instances, the calcium and phosphate reactants include one or more calcium containing compounds and one or more phosphate containing compounds. In some instances, the reactants include two or more phosphate containing compounds. In some instances, the reactants include two or more calcium containing compounds.

Calcium sources of interest include, but are not limited to: calcium carbonate (CaCO₃), calcium oxide (CaO), calcium hydroxide (Ca(OH)₂) and the like. Phosphate sources of interest include, but are not limited to: phosphoric acid (H₃PO₄), all soluble phosphates, and the like.

In some instances, the calcium and phosphate reactants include one or more compounds that include calcium and phosphate (e.g., a calcium phosphate). Calcium phosphate sources of interest that may be present in the dry reactants include: MCPM (monocalcium phosphate monohydrate or Ca(H₂PO₄)₂/H₂O); DCPD (dicalcium phosphate dihydrate, brushite or CaHPO₄.2H₂O), ACP (amorphous calcium phosphate or Ca₃(PO₄)₂H₂O), DCP (dicalcium phosphate, monetite or CaHPO₄), tricalcium phosphate, including both α- and β-(Ca₃(PO₄)₂, tetracalcium phosphate (Ca₄(PO₄)₂O, etc. In certain embodiments, wherein a calcium phosphate compound is employed, the ratio of calcium to phosphate (i.e., ratio of calcium cations to phosphate groups) of the compound ranges from 1 to 2.

In some instances, the subject compositions include a calcium or phosphate reactant (a first reactant) having a mean particle size (as determined using the Horiba LA-300 laser diffraction particle sizer (Version 3.30 software for Windows 95)(Irvine, Calif.)) of 8 μm or less and a narrow particle size distribution. The mean particle size of this reactant may vary, ranging in certain embodiments from 1 to 7 μm, from 1 to 6 μm, or from 1 to 5 μm, where the mean particle size in certain embodiments may be 1, 2, 3 and 4 μm. In some instances, the mean particle size is 3 μm.

In certain instances, this particular reactant (first reactant) of the subject compositions is further characterized in that it has a narrow particle size distribution. By narrow particle size distribution is meant that the standard deviation of the particles that make up the particular reactant population (as determined using the Horiba LA-300 laser diffraction particle sizer (Version 3.30 software for Windows 95)(Irvine, Calif.)) does not exceed 4.0, and, in certain embodiments, does not exceed 3.0, e.g., does not exceed 2.5, including does not exceed 2.0 μm.

This particular reactant (first reactant) of the subject compositions is further characterized in that the mode (as determined using the Horiba LA-300 laser diffraction particle sizer (Version 3.30 software for Windows 95)(Irvine, Calif.)) is 8.0 or less, and in certain embodiments is 6.0 or less, e.g., 5 or less, including 3.0 μm or less.

In certain embodiments, the calcium and phosphate reactants are further characterized by including a second reactant (e.g., a compound containing calcium, phosphate, or calcium and phosphate) that has a mean particle size that is 2 times or more larger than the mean particle size of the first reactant component, where the mean particle size of this second reactant is 9 μm or more, 10 μm or more, 20 μm or more, 25 μm or more, 30 μm or more larger (as determined using the Horiba LA-300 laser diffraction particle sizer (Version 3.30 software for Windows 95) (Irvine, Calif.)).

In certain embodiments, the amount of the first reactant is greater than the total amount of other reactants that are present, for example, the second reactant as described above. In these embodiments, the mass ratio of the first reactant to the total mass of the calcium and phosphate reactants ranges from 1 to 10, e.g., from 9 to 6, such as from 9 to 7, including from 9.5 to 8.5.

In certain embodiments, the first reactant is a calcium phosphate compound having a calcium to phosphate ratio ranging from 1.0 to 2.0, including from 1.33 to 1.67, such as 1.5. In certain embodiments, the calcium phosphate compound is a tricalcium phosphate, such as α- and β-tricalcium phosphate or a combination thereof. In certain embodiments, the tricalcium phosphate is α-tricalcium phosphate. In certain embodiments, the tricalcium phosphate is β-tricalcium phosphate. The first reactant, as described above, may be prepared using any convenient protocol, including by using an air pulverization protocol.

The ratios or relative amounts of each of the disparate calcium and/or phosphate compounds in the calcium and phosphate reactants is one that provides for the desired calcium phosphate product upon combination with an aqueous medium (e.g., fluid in vivo environment) and subsequent setting.

In some embodiments, the overall ratio (i.e., of all of the disparate calcium and/or phosphate compounds in the calcium and phosphate reactants) of calcium to phosphate in the calcium and phosphate reactants ranges from 4:1 to 0.5:1, from 2:1 to 1:1 or from 1.9:1 to 1.33:1.

The amount of calcium and phosphate reactants in the composition may vary. In some instances, the amount ranges from 10 to 99 wt %, such as 10 to 90 wt %, including 25 to 80 wt %.

As indicated above, the subject compositions also include a non-aqueous liquid carrier, wherein the calcium and phosphate reactants are suspended in the non-aqueous liquid carrier. In certain embodiments, the non-aqueous liquid carrier is a viscous liquid that contributes to the malleability of the subject composition. While the viscosity of the liquid may vary, in some instances the viscosity may range from 1 to 25k cps.

Any suitable non-aqueous liquid carrier that enhances the malleability of the subject composition may be used. In certain embodiments, the non-aqueous liquid carrier is a polyol. Polyols that can be used in the subject compositions include, but are not limited to: a glycol, a glycerol, a pentaerythritol, trimethylolethane, a propanediol, trimethylolpropane, 1,2,6-hexanetriol, sorbitol, inositol, dextran, a polylactic acid and a polyalcohol. Other non-aqueous liquid carrier that can be used in the subject compositions include, but are not limited to, polyvinylpyrolidon (PVP), propanol, a glycosaminoglycan (GAG), chondroitin sulfate, polyglycolic acid (PGA) and polylacticglycolic acid (PLGA). In some embodiments the non-aqueous liquid carrier is a polyalkylene oxide.

In some embodiments, the polyalkylene oxide is polyethylene glycol (PEG). In certain embodiments, the polyethylene glycol (PEG) has a molecular weight of 200 to 1,000. In certain embodiments, the PEG has a molecular weight of 250 to 1,000, 300 to 1,000, 350 to 1,000, 400 to 1,000, 450 to 1,000, 500 to 1,000, 550 to 1,000, 600 to 1,000, 650 to 1,000, 700 to 1,000, 750 to 1,000, 800 to 1,000, 850 to 1,000, 900 to 1,000 or 950 to 1,000 daltons. In certain embodiments, the PEG has a molecular weight of 200 to 950, 200 to 900, 200 to 850, 200 to 800, 200 to 750, 200 to 700, 200 to 650, 200 to 600, 200 to 550, 200 to 500, 200 to 450, 200 to 400, 200 to 350, 200 to 300 or 200 to 250 daltons. In other embodiments, the PEG has a molecular weight of 200 to 300, 250 to 350, 300 to 400, 350 to 450, 400 to 500, 450 to 550, 500 to 600, 550 to 650, 600 to 700, 650 to 750, 700 to 800, 750 to 850, 800 to 900, 850 to 950 or 900 to 1000 daltons. In yet other embodiments, the PEG has a molecular weight of 25 or more, 50 or more, 75 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 950 or more, 1,000 or more, 1,250 or more, 1,500 or more, 1,750 or more, 2,000 or more, 2,500 or more or 3,000 or more daltons.

The amount of non-aqueous liquid carrier in the composition may vary. In some instances, the amount ranges from 1 to 50, such as 5 to 40, including 10 to 30 wt %.

In certain embodiments, the subject malleable calcium phosphate compositions further include a monovalent cation dihydrogen phosphate salt. By monovalent cation dihydrogen phosphate salt is meant a salt of a dihydrogen phosphate anion and a monovalent cation, e.g., K+, Na+, etc., where the salt may or may not include one or more water molecules of hydration, e.g., may be anhydrous, a monohydrate, a dihydrate, etc. The monovalent cation dihydrogen phosphate salts present in the compositions of the subject invention may be described by the following formula:

Y⁺H₂PO₄.(H₂O)ₙ

where:

Y⁺ is a monovalent cation, such as K+, Na+, etc.; and

n is an integer from 0 to 2.

In certain embodiments, the salt is a sodium dihydrogen phosphate salt, such as sodium biphosphate (i.e., sodium phosphate monobasic, NaH₂PO₄), or the monohydrate (NaH₂PO₄.H₂O) or dihydrate (NaH₂PO₄.2H₂O) thereof.

The amount of monovalent cation dihydrogen phosphate salt that is present in the reactants may vary, and may present in an amount sufficient to provide for a rapidly setting high strength attainment composition. In some embodiments, the salt is present in an amount that ranges from 0.10 to 20 wt. %, such as from 0.2 to 15.0 wt %, including from 1 to 10.0 wt. % of the total weight of the reactants.

In certain embodiments, the compositions may further include an amount of an emulsifying agent, as described in U.S. Application Publication No. 2005/0260279; the disclosure of which is herein incorporated by reference. Emulsifying agents of interest include, but are not limited to: polyoxyethylene or polyoxypropylene polymers or copolymers thereof, such as polyethylene glycol and polypropylene glycol; nonionic cellulose ethers such as methylcellulose, ethylcellulose, hydroxymethylcellulose, hydroxyethylcellulose, carboxymethylcellulose, carboxyethylcellulose and hydroxypropylcellulose; additional celluloses, such as carboxymethylcellulose sodium, carboxymethylcellulose calcium, carboxymethylstarch; polysaccharides produced by microbial fermentation, such as yeast glucans, xanthan gum, β-1,3-glucans (which may be straight-chained or branched; e.g. curdlan, paramylum, pachyman, scleroglucan, laminaran); other natural polymers, e.g., gum arabic, guar gum, carrageenin, gum tragacanth, pectin, starch, gelatin, casein, dextrin, cellulose; polyacrylamide; polyvinyl alcohol; starch; starch phosphate; sodium alginate and propylene glycol alginate; gelatin; amino-containing acrylic acid copolymers and quaternization products derived therefrom; and the like. The amount of monovalent cation dihydrogen phosphate salt that is present in the reactants may vary. In some embodiments, the emulsifier is present in an amount that ranges from 0.01 to 10 wt. %, such as from 0.01 to 5.0 wt %, including from 0.1 to 1.0 wt. % of the total weight of the reactants.

The subject calcium phosphate compositions may further include one or more other components that modulate the properties of the product calcium phosphate mineral containing product formed by the subject compositions. In instances wherein the subject composition forms a bioresorbable product, the component is released at the site of implantation upon resorption of the product. Such additional components include, but are not limited to: organic polymers, e.g., proteins, including bone associated proteins which impart a number of properties, such as enhancing resorption, angiogenesis, cell entry and proliferation, mineralization, bone formation, growth of osteoclasts and/or osteoblasts, and the like, where specific proteins of interest include, but are not limited to: osteonectin, bone sialoproteins (Bsp), α-2HS-glycoproteins, bone Gla-protein (Bgp), matrix Gla-protein, bone phosphoglycoprotein, bone phosphoprotein, bone proteoglycan, protolipids, bone morphogenic protein, cartilage induction factor, platelet derived growth factor, skeletal growth factor, and the like; particulate extenders; inorganic water soluble salts, e.g., NaCl, calcium sulfate; sugars, e.g., sucrose, fructose and glucose; pharmaceutically active agents, e.g., antibiotics; and the like. Additional active agents of interest include osteoclast induction agents, e.g., RANKL, as described in U.S. Pat. No. 7,151,833, the disclosure of which is herein incorporated by reference. In certain embodiments, the component is a component that promotes bone growth, e.g., an osteoinductive factor. Osteoinductive factors include, for example, bone morphogenetic growth factors (BMPs, e.g., BMP-2 and BMP-7), IGF, TGF-β, IGFs (IGF-1 and IGF-2), parathyroid hormone, angiogenic factors, osteocalcin and osteopontin.

In some embodiments, the compositions include a demineralized bone matrix (DBM). As used herein, a “demineralized bone matrix” and “DBM” refer to a bone matrix composition wherein a portion or all inorganic mineral content is removed.

Demineralized bone matrix may be obtained from a commercial source or prepared by any suitable protocol. For example, demineralized bone matrix may be formed by decalcification of cortical or cancellous bone by acid extraction. Acid solutions that may be utilized for demineralizing bone matrices include, but are not limited to, inorganic acids, such as hydrochloric acid and organic acids such as formic acid, acetic acid, peracetic acid, citric acid and propionic acid. Exemplary protocols for demineralizing bone matrices are disclosed in U.S. Pat. Nos. 5,073,373; 5,290,0555; 484,601; 5,284,655, Reddi et al. Proc. Natl. Acad. Sci. 69: 1601-1605 (1972); Urist et al., Proc Natl Acad Sci U.S.A. 81:371-375 (1984); and Lewandrowski et al. J. Biomed Mater. Res. 31: 365-372 (1996), the disclosures of which are incorporated herein by reference. Exemplary commercial sources of DBM include for instances, Regeneration Technologies Inc. (Alachua, Fla., USA) and The American Red Cross (Arlington, Va., USA). In some instances, the source of the demineralized bone matrix is cortical bone. In other instances, the source of the demineralized bone matrix is cancellous bone. In yet other instances, the source of the demineralized bone matrix is a combination of cortical and cancellous bone. The demineralized bone matrix of the subject compositions may be derived from the shaft of long bones or from flat bone structures, such as, for example, a skull. Moreover, the DBM may be derived from the outer periosteal layer, the middle layer or the inner endosteal layer or combinations thereof (e.g., derived from the periosteal layer and the middle layer) of a particular bone. Demineralized bone matrix used in the subject compositions may be an autologous, allogeneic, or xenogenic demineralized bone matrix.

The degree of demineralization of a particular bone matrix may depend on the shape of the bone being demineralized, the strength and temperature of the acid solution, the bone source that is being demineralized, the degree of agitation of the bone matrix, and the extent of demineralization. Demineralized bone matrices used in the subject implantable compositions include partially demineralized bone matrices. In certain embodiments, the demineralized bone matrix is a bone matrix that contains 99%, 95%, 90%, 85%, 80%, 75%, 70%, 65%, 60%, 55%, 50%, 45%, 40%, 35%, 30%, 25%, 20%, 15%, 10%, 5%, 1% or less of its original inorganic mineral content.

Demineralized bone matrices used in the subject compositions may also have been subjected to a defatting and/or disinfecting step prior to demineralization. Any suitable solution may be used for defatting/disinfecting of a bone sample prior to demineralization. In certain instances, the defatting/disinfecting step is carried out in the presence of an aqueous ethanol solution. Other defatting/disinfecting agents include, but are not limited to, methanol, propanol, isopropanol, butanol, denatured ethanol, DMF, DMSO, diethyl ether, hexanes, glyme, tegrahydrofuran, chloroform, methylene chloride and carbon tetrachloride.

Demineralized bone matrices used in the subject compositions may have osteoinductive and/or osteoconductive properties. Osteoinductive demineralized bone matrices contain one or more osteoinductive factors such as, for example, bone morphogenetic growth factors (BMPs, e.g., BMP-2 and BMP-7), IGF, TGF-β, IGFs (IGF-1 and IGF-2), parathyroid hormone, angiogenic factors, osteocalcin and osteopontin. In certain instances, the demineralized bone matrix includes one or more osteoinductive factors. In certain embodiments, the subject composition is osteoinductive. In other embodiments, the subject composition is osteoconductive. In yet other embodiments, the subject composition is osteoinductive and osteoconductive.

Demineralized bone matrices used in the subject compositions may also contain collagen that has undergone further processing. In some embodiments, the collagen of the demineralized bone matrix included in the subject composition is chemically modified to achieve a polyanionic collagen. See, e.g., Bet et al. Biomacromolecules 2: 1074-1079 (2001). Polyanioinic collagen can improve cell adhesion as compared to the native collagen counterpart. In some instances, the collagen is purified to remove one or more antigenic components. For example, in some embodiments, collagen used in the subject compositions is treated to remove antigenic telopeptide regions. Purification of collagen to remove such antigenic components can be carried out by enzymatic digestion (e.g., pepsin digestion). See, e.g., Rovira et al. Biomaterials 17: 1535-1540 (1996). In certain embodiments, a portion of the collagen of the DBM is removed. Removal of collagen from a DBM can be accomplished, for example, using collagenase, mechanical treatment or heat treatment.

In some instances, the collagen of the subject composition is cross-linked collagen. Cross-linking collagen can increase the mechanical strength, thermal stability, and resistance to proteolytic breakdown of collagen in the compositions provided herein. The collagen may be cross-linked by any suitable protocol including, but not limited to, by using a chemical agent, physical heating, UV irradiation, or a combination of such protocols. See, e.g., Chau et al. Biomaterials 26(33): 6518-6529 (2005), the disclosure of which is incorporated herein by reference. In some embodiments, the collagen of the subject sponge composition is chemically cross-linked. Any suitable protocol may be used to chemically cross-link collagen, including, but not limited to, those described in Rault et al. J Mater Sci Mater Med 7:215-221 (1996). Exemplary agents for chemically cross-linking collagen include, but are not limited to, glutaraldehyde (GTA), formaldehyde, genipin, hexamethylene diisocyanate (HMDC), cyanamide, 1-ethyl-3-(3-dimethyl aminopropyl) carbodiimide (EDC) and acyl azide (e.g., hydrazine and diphenylphosphorylazied (DPPA)). In certain embodiments, the collagen of the subject compositions is glutaraldehyde cross-linked collagen.

In some instances, the demineralized bone matrix includes one or more agents that aid in the repair of a bone defect. In certain instances, the one or more agents are an agent not found in naturally occurring bone matrices. In certain instances, the one or more agents is an agent that is present in the demineralized bone matrix at a greater level as compared to its level in naturally occurring bone matrices. In other embodiments, the agent(s) is an agent that is present in the demineralized bone matrix at a lesser level as compared to its level in naturally occurring bone matrices. Exemplary agents include, but are not limited to, antiviral drugs, antibiotics, antimicrobial drugs, growth factors (e.g., osteoinductive or osteogenic factors), and immunosuppresants. Such an agent may be introduced into the DBM by soaking the DBM in the agent and subsequent drying of the DBM.

Upon contact with an aqueous medium (e.g., an in vivo biological fluid), the calcium and phosphate reactants of the subject calcium phosphate compositions react to form calcium phosphate mineral containing products. By “calcium phosphate mineral containing” product is meant a solid product that includes one or more calcium phosphate minerals. In certain instances, the calcium phosphate mineral of the product is different than the calcium and phosphate reactants of the subject composition. In certain embodiments, the calcium phosphate mineral is one that is generally poorly crystalline, so as to be resorbable and, often, remodelable, over time when implanted into a physiologically site. The calcium to phosphate ratio in the product may vary depending on particular reactants and amounts thereof employed to produce it. In certain embodiments, the calcium to phosphate ratio is 2:1 to 1.33:1. In some instances, the calcium to phosphate ratio is 1:7:1 to 1.6:1. In certain embodiments, the calcium and phosphate reactants react to form apatitic products. Apatitic products have a calcium to phosphate ratio ranging from 2.0:1 to 1.33:1, including both hydroxyapatite and calcium deficient analogs thereof, including carbonate substituted hydroxyapatite (i.e., dahllite), etc. The subject malleable calcium phosphate composition is, in certain embodiments, one that is capable of forming a hydroxyapatitic product.

The period of time required for the calcium phosphate compositions to form the calcium phosphate mineral-containing product upon contact with an aqueous medium may vary. In some instances, this time varies from 10 min to 24 hrs. In some embodiments, the calcium phosphate composition forms the calcium phosphate mineral-containing product in a clinically relevant period of time. By clinically relevant period of time is meant that the calcium phosphate composition sets in 20 minutes or less, where the composition remains malleable for 1 minute or more, 2 minutes or more or 5 minutes or more, following the presence of the aqueous medium.

In some instances, the subject calcium phosphate compositions rapidly form a high strength product, as determined by the Gilmore Needle Test (ASTM C266-89) in terms of setting value. More specifically, the compositions attain high strength rapidly, such that they may be viewed as rapid strength attainment compositions. As such, at 3 minutes the compositions have a setting value of 200 Newtons or more, such as 300 Newtons or more, where the setting value may be as high as 400, 500, 600 or more Newtons. At 6 minutes the compositions have a setting value of 400 Newtons or more, such as 500 Newtons or more, where the setting value may be as high as 600, 700, 800, 900, 1000 or more Newtons.

In certain instances, the subject compositions are malleable prior to contact with an aqueous medium as well as while they are forming the calcium phosphate mineral containing product. As such, they may be manipulated as the calcium and phosphorous reactants react to form the calcium phosphate mineral-containing product, without adversely affecting the properties of the final product. For example, during formation of the final calcium phosphate mineral-containing product, screws can be drilled into them, without adversely impacting the properties of the final product.

In certain embodiments, the subject compositions are characterized as compositions that go through the following phases: (1) a setting phase, in which the composition should be maintained without manipulation; (2) a “drillable” phase, in which hardware, such as screws, may be inserted or positioned into the composition; and (3) a screw tightening phase, in which screws positioned in the composition during the “drillable” phase may be tightened without adversely affecting the composition. In certain embodiments, the setting phase ranges in duration from 1 minute to 15 minutes, e.g., from 1 minutes to 10 minutes following contact with an aqueous medium. In certain embodiments the drillable phase commences from 5 minutes to 10 minutes following contact with an aqueous medium, and may extend to 10 minutes to 15 minutes or longer following contact with an aqueous medium. In certain embodiments, the screw tightening phase ranges comments from 10 minutes to 15 minutes following contact with an aqueous medium.

The compressive strength of the final calcium phosphate mineral containing product formed by the subject composition may vary significantly depending on the particular components employed to produce it. In some embodiments, the composition produces a product that has a compressive strength sufficient for it to serve as at least a cancellous bone structural material. By cancellous bone structural material is meant a material that can be used as a cancellous bone substitute material as it is capable of withstanding the physiological compressive loads experienced by compressive bone under at least normal physiological conditions. As such, the subject composition is one that forms a calcium phosphate mineral containing product having a compressive strength of 20 or more, 40 or more, or 50 or more MPa, as measured by the assay described in Morgan, E F et al., 1997, Mechanical Properties of Carbonated Apatite Bone Mineral Substitute: Strength, Fracture and Fatigue Behavior. J. Materials Science: Materials in Medicine. V. 8, pp 559-570, where the compressive strength of the final product may be as 60 MPa or more. Compressive strengths can be obtained that range from 100 to 200 MPa. In certain embodiments, the resultant product has a tensile strength of 0.5 MPa or more, such as 1 MPa or more, including 5 MPa or more, and 10 MPa.

In certain instances, the resultant calcium phosphate mineral containing product is stable in vivo for extended periods of time, by which is meant that it does not dissolve or degrade (exclusive of the remodeling activity of osteoclasts) under in vivo conditions, e.g., when implanted into a living being, for extended periods of time. In these embodiments, the resultant product is stable for 4 months or longer, 6 months or longer up to 1 year or longer. In certain embodiments, the resultant product is stable in vitro when placed in an aqueous environment for extended periods of time, by which is meant that it does not dissolve or degrade in an aqueous environment, e.g., when immersed in water, for extended periods of time. In these embodiments, the resultant product is stable for 4 months or longer, 6 months or longer, or 1 year or longer, e.g., 2.5 years, 5 years, etc.

In other instances, the resultant calcium phosphate mineral-containing product is bioresorbable. Bioresorbable products formed by the subject compositions can be broken down and assimilated in a subject (e.g., a human subject). As such, bioresorbable products allow for a temporary scaffold for tissue growth (e.g., hard tissue) or for the delivery of an agent in a subject. A subject composition that forms a bioresorbable calcium phosphate mineral-containing product is capable of remaining in a subject for a sufficient amount of time for its intended purpose (e.g., to allow for tissue growth and/or delivery of an effective amount of a therapeutic agent). In certain instances, the calcium phosphate mineral containing product form by the subject composition remains in a subject for 1, 2, 3, 4, 5, 6, 7, 8, 9, 10, 11, 12 weeks or less.

### Methods of Making Implantable, Malleable Calcium Phosphate Compositions

In another aspect provided herein is a method of making an implantable and malleable calcium phosphate composition, for example, any one of the subject calcium phosphate compositions provided herein. In certain embodiments, the method includes combining calcium and phosphate reactants with a non-aqueous liquid carrier. In practicing the subject methods, suitable amounts of the calcium and phosphate reactants and non-aqueous liquid carrier are combined to produce a malleable calcium phosphate composition. In certain instances, the liquid to solids ratio is chosen to provide for a malleable composition that has a viscosity ranging from that of milk to that of modeling clay. As such, in certain instances, the non-aqueous liquid carrier to solids ratio employed in the subject methods ranges from 0.1 to 2.0.

As mentioned above, the requisite amounts of calcium and phosphate reactants and non-aqueous liquid carrier are combined under conditions sufficient to produce the subject composition. As such, the calcium and phosphate reactants and non-aqueous liquid carrier are typically combined under agitation or mixing conditions, such that a homogenous suspension is produced from the reactants and liquid components. Mixing may be accomplished using any convenient means, including manual or automated mixing. The above-described protocols result in the production of a implantable and malleable calcium phosphate composition that is capable of forming a calcium phosphate containing product in the presence of an aqueous medium (e.g. a fluid in vivo environment), as described in greater detail above.

### Utility

The subject compositions, e.g., as described above, find use in applications where it is desired to introduce a composition capable of forming into a solid calcium phosphate product into a physiological site of interest, such as in dental, craniomaxillofacial and orthopedic applications. In the absence of an aqueous medium, the calcium and phosphate reactants of the malleable calcium phosphate composition do not react. In the presence of an aqueous medium (e.g., a fluid in vivo environment upon implantation in a subject), however, the calcium and phosphate reactants react, resulting in a calcium phosphate containing product not found in the original reactants. As such, the subject malleable calcium phosphate compositions provided herein do not require the addition of any other components or mixing prior to use and can be stored as a single composition.

In orthopedic applications, the subject composition may be prepared, as described above, and introduced to a bone repair site, such as a bone site comprising cancellous and/or cortical bone. Other orthopedic applications in which the subject compositions find use include, but are not limited to, the treatment of fractures and/or implant augmentation, in mammalian hosts, particularly humans. In such fracture treatment methodologies, the fracture is first reduced. Following fracture reduction, a subject composition is introduced into the cancellous tissue in the fracture region using an application (e.g., a spatula or syringe). Specific dental, craniomaxillofacial and orthopedic indications in which the subject invention finds use include, but are not limited to, those described in U.S. Pat. No. 6,149,655, the disclosure of which is herein incorporated by reference. In addition to these particular applications described in this U.S. Patent, the subject calcium phosphate compositions also find use in applications where a sternotomy has been performed. Specifically, the subject compositions find use in the closure process of a sternotomy, where the bone fragments are rejoined and wired together, and any remaining cracks are filled with the subject composition. In yet other embodiments, the subject compositions find use in drug delivery, where they are capable of acting as long lasting drug depots following administration to a physiological site. See e.g., U.S. Pat. Nos. 5,904,718 and 5,968,253; the disclosures of which are herein incorporated by reference.

In certain embodiments, the subject compositions may also be seeded with any of a variety of cells prior to application at a physiological site of interest. Cells that may be seeded include those described in published U.S. Pat. Nos. 6,139,578 and 7,252,833, the disclosures of which is herein incorporated by reference. Cells that may be seeded in the subject composition include tissue forming cells, bone forming cells, cartilage forming cells and tissue degrading cells. Exemplary cells that may be seeded with the subject composition prior to application include, but are not limited to: chondrocytes, osteocytes, osteoblasts, osteoclasts, mesenchymal cells, and fibroblasts.

Representative applications of interest also include, but are not limited to: those described in U.S. Pat. Nos. 6,375,935; 6,719,993; 7,252,672; 7,252,833; 7,252,841; 7,261,717; and 7,306,786, the disclosures of which are herein incorporated by reference.

### Kits

Also provided are kits that include the subject malleable calcium phosphate composition. Of interest are kits that include a sterile package, e.g., a pouch, envelope, rigid sealed container, etc., that includes an amount of the malleable calcium phosphate composition. In addition to the calcium phosphate composition, the subject kits may further include an applicator, such as but not limited to: a spatula, a catheter, or a syringe, etc.

In addition to above-mentioned components, the subject kits typically further include instructions for using the components of the kit to practice the subject methods. The instructional material may also be instructional material for using the subject malleable calcium phosphate compositions, e.g., it may provide surgical techniques and principles for a particular application in which the composition is to be employed. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., flash drive, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

### Example I

### 11.1. Chemical Composition of Device

Testing of an implantable malleable calcium phosphate composition falling within the scope of the claims of this application (hereinafter referred to as “Skaffold NMX”) was performed as outlined in the Class II Special Controls Guidance Document: Resorbable Calcium Salt Bone Void Filler Device; Guidance for Industry and FDA issued on Jun. 2, 2003.

11.1.1. Identification of Device Material

The Skaffold NMX composition consists of a mixture of calcium phosphate powder (i.e., α-tricalcium phosphate particles, SMPA and CMC as described in the Experimental Section of U.S. patent application Ser. No. 12/771,999, the disclosure of which is herein incorporated) in a bioinert polyethylene glycol (PEG) based polymer. The amounts of each component present in the device are mentioned in Table 1. No other phases (crystalline or amorphous), additives, binders or adjuncts are present.

TABLE 1
Weight percent of components present in the device

| | Weight |
|---|---|
| Material | Percent |
| Calcium Phosphate | 62.2 |
| SPMA | 9.6 |
| CMC | 0.1 |
| PEG | 28.0 |

The calcium to phosphorus (Ca/P) ratio is 1.5.

11.1.2. Identification of the Crystalline and Non-Crystalline Phases, Phase Purity and Weight Percentage of Phases

All phases present in the device after curing are crystalline with no detectable amorphous phases as confirmed by Fourier Transform Infrared Spectroscopy (FTIR) and X-ray Diffraction (XRD). The procedures and results for both these tests are as follows:

### A. Fourier Transformed Infrared Spectroscopy

### Procedure

Skaffold NMX was filled into the molds (Internal Diameter=0.5 inches and Height=0.3 inches) and cured in PBS at 37° C. for 72 hours. After the first 24 hours, the cement were removed from the molds and placed back in the same PBS solution at previous conditions. Afterwards, the cured cement was dried at 37° C. for 72 hours. The dried cement was ground in an agate mortar and sample pellet was made by mixing 1 mg of the dried powder with 300 mg of dried spectroscopic grade KBr and compressed in a vacuum die under a pressure of 50,000 psi. The pellet was run on a FTIR machine (Nicolet iS10, Thermo-Nicolet, Woburn, Mass.) at 64 scans at a typical resolution of 4 cm⁻¹.

### Results

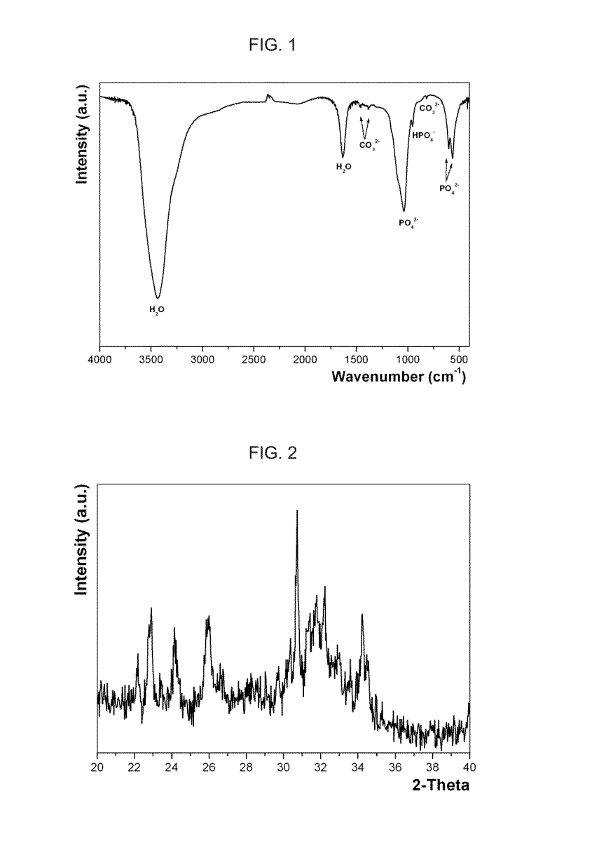

FIG. 1 displays the FTIR pattern of Skaffold NMX following in vitro incubation for 72 hours. The FTIR pattern is displayed from 4000 to 400 cm⁻¹, and depicts the different bands present in the spectra. The PO₄²⁻ bands are present at 1035, 602 and 563 cm⁻¹ and correspond to apatite. The appearance of absorption band at 950 cm⁻¹ demonstrates incorporation of HPO₄⁻ group in the structure of apatite and the presence of absorption bands at 1452, 1416 and 870 cm⁻¹ confirms substitution of carbonate (CO₃²⁻) groups for phosphate. These results indicate low crystalline order apatite that resembles inorganic phase of bone with no evidence of peaks corresponding to other mineral phases.

### B. X-Ray Diffraction (XRD)

### Procedure

Skaffold NMX putty was filled into the molds (Internal Diameter=0.5 inches and Height=0.3 inches) and cured in PBS at 37° C. for 72 hours. After the first 24 hours, the cement were removed from the molds and placed back in the same PBS solution at previous conditions. Afterwards, the cured cement was dried at 37° C. for 72 hours. The dried cement was ground in an agate mortar and XRD powder diffraction sample was prepared. An Ultima IV powder X-ray diffractometer (Rikagu USA, The Woodlands, Tex.), operated at 40 kV and 44 mA, equipped with a Copper (Cu) tube was utilized to collect X-ray Diffraction patterns for these powder samples. X-ray diffraction data was collected for 28 values of 20° to 40° with a step size of 0.02° 28 and a preset time of 1 second at each step.

### Results

FIG. 2 displays the XRD pattern of Skaffold NMX following in vitro incubation for 72 hours. The diffraction pattern also shows predominantly hydroxyapatite of low crystalline order (09-0432) with evidence of small amounts of the starting reactants alpha-tricalcium phosphate (09-0348). The amount of hydroxyapatite present is 70% and alpha-tricalcium phosphate is present in 30% following in vitro incubation for 72 hours.

11.1.3. Elemental Analysis

The heavy metal/trace elemental analysis was performed using inductively coupled plasma-mass spectroscopy (ICP-MS). The procedure and results for the test are described below:

### Procedure

Skaffold NMX was filled into the molds (Internal Diameter=0.5 inches and Height=0.3 inches) and cured in PBS at 37° C. for 72 hours. After the first 24 hours, the cement were removed from the molds and placed back in the same PBS solution at previous conditions. Afterwards, the cured cement was dried at 37° C. for 72 hours and ground into fine powder. Chemical analysis of dried Skaffold NMX powder was performed by ICP-MS. For analyzing, 0.2 g weighed powder portion was dissolved in a solution consisting of 30 mL water, 1 mL nitric acid, 3 mL hydrochloric acid and an internal standard solution. This solution was further diluted to 100 mL and analyzed using for ICP-MS analysis. Duplicate tests were run for the sample in order to confirm the prior results.

### Results

The ASTM standard F1185-03 (2009) suggested a limit to heavy metals/trace elements presence as presented in Table 2 below. Skaffold NMX contained substantially lower heavy metal/trace elements than limits described in the ASTM Standard.

TABLE 2
Heavy Metal/Trace Elements in Skaffold NMX versus Callos

| Element | | |
|---|---|---|
| Other Metals | ASTM F1185-03 | Skaffold NMX |
| Pb—Lead | 50 | ND |
| Hg—Mercury | 5 | ND |
| As—Arsenic | 3 | ND |
| Cd—Cadmium | 5 | ND |

*ND—Not Detected

Table 3 below lists trace elements that are present in Skaffold NMX above 100 ppm. These elements have no applicable standard limits.

TABLE 3
Other Elements Detected Above 100
ppm in Skaffold NMX and Callos

| Element | Amount (ppm) - |
|---|---|
| Other Metals | Skaffold NMX |
| Fe—Iron | 108 |
| Mg—Magnesium | 638 |
| Na—Sodium | 18500 |
| Sr—Strontium | 209 |

Trace metal levels in Skaffold NMX meet the specifications listed in ASTM standard.

11.1.4. Diffraction Pattern Along with Superimposed Patterns of Each Phase as Given for the Relevant Calcium Salt Available from the International Center for Diffraction Data/Joint Committee on Powder Diffraction Standards (ICDD/JCPDS)

All phases present in the submitted device are mentioned below with no other detectable crystalline or amorphous phase(s) present:
1. Alpha-tricalcium phosphate (α-TCP, Ca₃(PO₄)₂) phase purity >93% with an acceptable limit of up to 7% hydroxyapatite (HA, Ca₁₀(PO₄)₆(OH)₂),
2. Sodium phosphate monobasic anhydrous (SPMA, NaH₂PO₄, >99% purity),
3. Polyethylene Glycol 300, amorphous (>99.9% purity).

The setting reaction of the Skaffold NMX starts after immersing in PBS leading to the formation of the end product—hydroxyapatite (HA). The procedure and results of conversion of Skaffold NMX into hydroxyapatite are described below:

### Procedure

Skaffold NMX was filled into the molds (Internal Diameter=0.5 inches and Height=0.3 inches) and cured in PBS at 37° C. for 72 hours. After the first 24 hours, the cement was removed from the mold and placed back in the same PBS solution at previous conditions. Afterwards, the cured cements were dried at 37° C. for 72 hours. The dried Skaffold NMX was ground in an agate mortar and sample was prepared for XRD powder diffraction. An Ultima IV powder X-ray diffractometer (Rikagu USA, The Woodlands, Tex.), operated at 40 kV and 44 mA, equipped with a Copper (Cu) tube was utilized to collect X-ray Diffraction patterns for these powder samples. X-ray diffraction data was collected for 28 values of 20° to 40° with a step size of 0.02° 28 and a preset time of 1 second at each step.

### Results

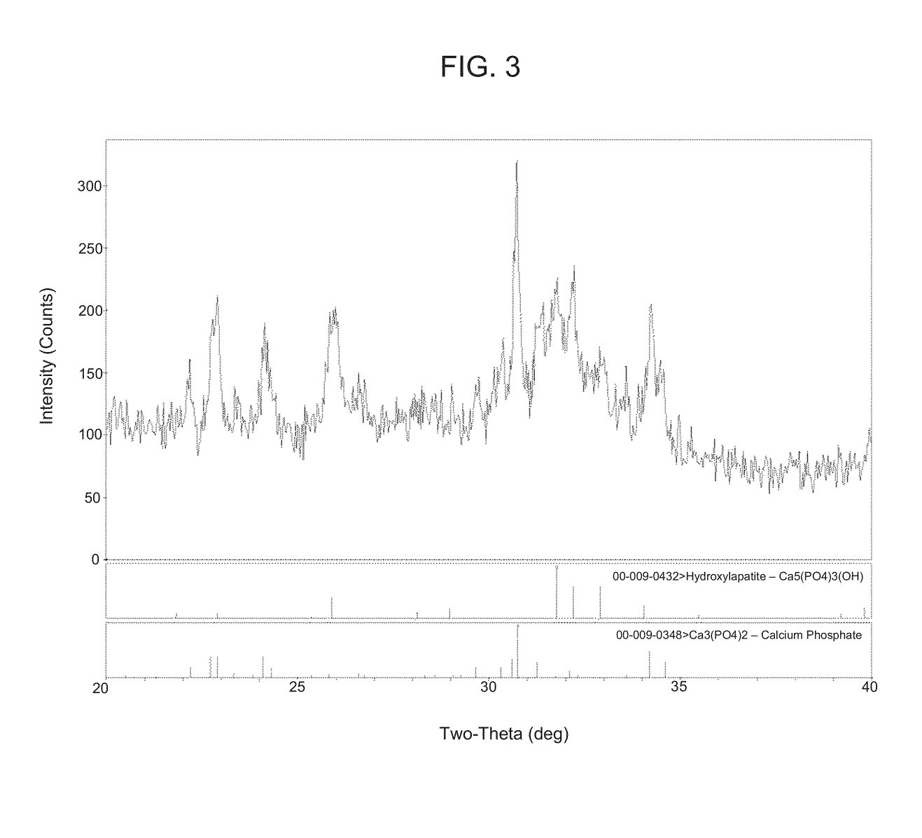

FIG. 3 shows x-ray diffraction patterns for Skaffold NMX following in vitro incubation for 72 hours. This diffraction pattern shows predominantly hydroxyapatite of low crystalline order with evidence of small amounts of crystalline starting reactant alpha-tricalcium phosphate. (ICDD #'s 09-432 and 09-0348, respectively). No other crystalline or amorphous phases were present.

### 11.2. Physical Properties of Device

11.2.1. Identification of the Device Physical Form

Skaffold NMX is available as a pre-mixed putty/paste intended to set in vivo. Scanning electron microscopy analysis was performed on fully cured Skaffold NMX to evaluate its microstructure upon curing.

### Procedure

Skaffold NMX was filled into the molds (Internal Diameter=0.5 inches and Height=0.3 inches) and cured in PBS at 37° C. for 72 hours. After the first 24 hours, the cement was removed from the mold and placed back in the same PBS solution at previous conditions. Afterwards, the cured cement was dried at 37° C. for 72 hours and after drying cracked into two pieces. Surface morphology of the cracked surface of sputter-coated (w/Pt) Skaffold NMX was evaluated with a scanning electron microscope (FE-SEM; FEI Quanta 650). The SEM was used in SE mode with an acceleration voltage of 10 kV.

### Results

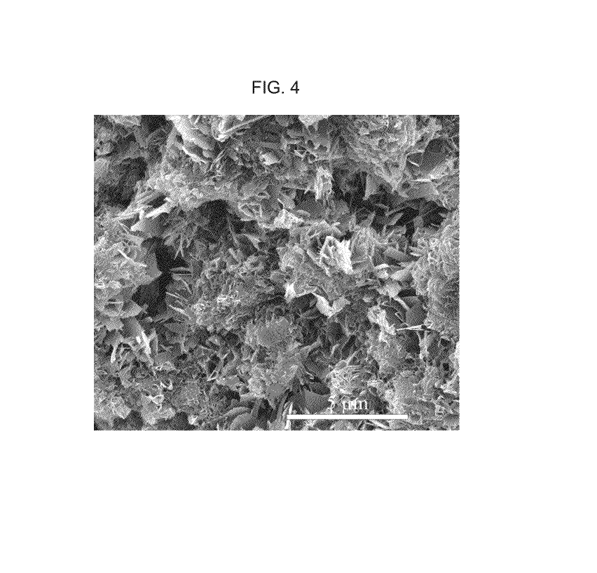

FIG. 4 provides an SEM micrograph of cured and dried Skaffold NMX, and shows inter-mingling, inter-locking, nano-sized crystals of hydroxyapatite. This morphology is typical morphology obtained in a hydroxyapatite-based self-setting bone void fillers.

11.2.2. Dimensional Specifications for all Device Configurations

### Procedure

Samples (0.1 g) of each fraction (fine, medium and coarse) of alpha tricalcium phosphate, were analyzed in 100% isopropanol using laser particle size analysis (Horiba LA950).

### Results

The particle size of different fractions of alpha-tricalcium phosphate is mentioned in Table 4.

TABLE 4
Mean particle size of various components of Skaffold NMX

| | Mean Particle Size | Appendix |
|---|---|---|
| Material | (μm) | Number |
| Fine alpha-tricalcium phosphate | 5.73 | B1 |
| Medium alpha-tricalcium | 84.02 | B2 |
| phosphate | | |
| Coarse alpha-tricalcium | 209.51 | B3 |
| phosphate | | |

11.2.3. Specification of Device Mass, Volume and Density

### Procedure

Skaffold NMX was filled into the molds (Internal Diameter=0.5 inches and Height=0.3 inches) and cured in PBS at 37° C. for 72 hours. After the first 24 hours, the cement were removed from the molds and placed back in the same PBS solution at previous conditions. Afterwards, the cured cement was dried at 37° C. for 72 hours. The apparent density was estimated by water displacement methods on cured and dried cements. The mass for both cements after curing was recorded prior to immersion in a known volume of PBS. Apparent density was calculated by dividing the weight in grams by its volume of material.

### Results

Representative data for six Skaffold NMX kits and the corresponding density measurements are shown in Table 5. The estimated density of Skaffold NMX following incubation in vitro in PBS at 37° C. for 72 hours is 1.79 grams/cc.

TABLE 5
Density Calculations for Skaffold NMX after curing
and drying in PBS at 37° C. for 72 hours

| | Weight | Start Volume | Displaced Volume | Volume | |
|---|---|---|---|---|---|
| | (g) | (cc) | (cc) | (cc) | Density |
| | 1.64 | 5.00 | 5.90 | 0.90 | 1.83 |
| | 1.65 | 5.00 | 5.90 | 0.90 | 1.83 |
| | 1.72 | 5.00 | 6.00 | 1.00 | 1.72 |
| | 1.80 | 5.00 | 6.00 | 1.00 | 1.80 |
| | 1.58 | 5.00 | 5.90 | 0.90 | 1.76 |
| | 1.63 | 5.00 | 5.90 | 0.90 | 1.81 |
| Average | 1.67 | | | 0.93 | 1.79 |

11.2.4. Specification of Device Porosity

Mercury intrusion porosimetry was used to calculate the specifications:
A. The total porous volume was 0.29 ml pores per gram Skaffold NMX.
B. The median pore diameter was 0.04 μm.
C. The device interconnected porosity or interconnectedness was reported as 41.1%.

### Example II

### Device Performance Testing

### Bench

### 18.1. pH Testing

### Testing Compared to Predicate Device

The purpose of this test is to compare the pH change of phosphate buffered saline (PBS) with Skaffold NMX versus Callos® calcium phosphate cement (Skeletal Kinetics, Cupertino Calif.).

### Procedure

Skaffold NMX was used as is and Callos was mixed according to the instructions for use. 5.0 g mixed cement pastes were placed at the bottom of a 100 ml glass bottle containing 100 ml of pH 7.4 Dulbecco's phosphate buffered saline (PBS). This glass bottle was placed in an incubator maintained at 37° C. and 5% CO₂. A calibrated pH probe with an ATC temperature compensator was placed into the PBS containing mixed cement and pH was measured at different time points—at start (0), 0.17, 0.5, 1, 2, 4, 24 and 48 hours after immersion. The procedure was performed in triplicates. PBS was used as control.

### Results

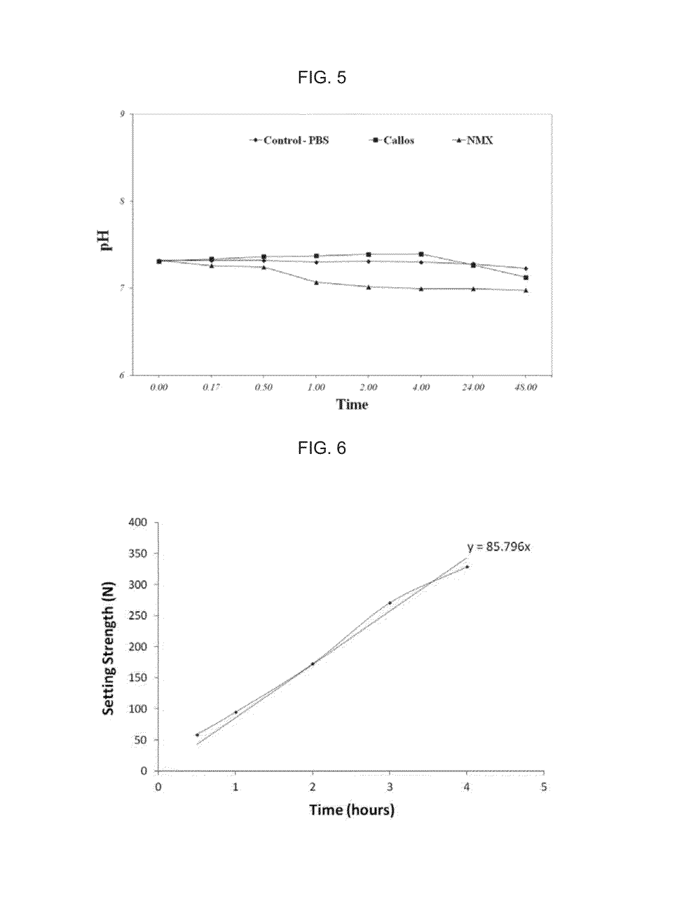

FIG. 5 presents the pH changes of the PBS with Skaffold NMX (average of N=3 samples). The data shows that there was negligible change in the pH of the PBS surrounding the Skaffold NMX. The pH of PBS with Callos and PBS alone (without any cement) were also examined and negligible changes were observed at the mentioned time points.

### 18.2. Dissolution/Solubility Testing

### Testing Compared to Predicate Device

The purpose of this test is to evaluate the dissolution behavior of subject device (Skaffold NMX) and compare with that of Callos® calcium phosphate cement and the values published in literature. Methods to determine dissolution rates in vitro were developed independently with reference to methods reported in the literature¹. No known published standards such as ASTM methods exist for these types of studies. ¹ P. Ducheyne, S. Radin, L. King, “The effect of calcium phosphate composition and structure on in vitro behavior: I. Dissolution,” Journal of Biomedical Materials Research, 27, 25 (1993).

### Procedure

Skaffold NMX was used as is and Callos® calcium phosphate cement was mixed according to the instructions for use. 5 grams of cement pastes—Skaffold NMX and Callos® calcium phosphate cement were immersed separately in 100 ml Dulbecco's PBS maintained at physiological conditions (pH=7.4, 37° C.) for 7 days with fresh PBS. The collected eluate was centrifuged to remove any solids from the liquid. This solution was then tested for Ca²⁺ concentration by inductively coupled plasma-atomic emission spectroscopy (ICP-AES) to determine the amount of calcium released during incubation of the Skaffold NMX and Callos. This study was performed in triplicates, and PBS served as the control.

### Results

The calcium ion concentration of the PBS containing Skaffold NMX and Callos was detected using ICP-AES. The results are presented in Table 6.

TABLE 6
Calcium ion concentration for Skaffold NMX, predicate
device and theoretical & literature values.

| Material | Calcium ion concentration (ppm) |
|---|---|
| Skaffold NMX | 6.45 |
| Predicate device - Callos ® | 5.45 |
| Calcium-Deficient Hydroxyapatite | 8.0-9.6 |
| (literature evidence)^{2,3} | |

²M. T. Fulmer, I. C. Ison, C. R. Hankermayer, B. R. Constantz, and J. Ross, “Measurements of the solubilities and dissolution rates of several hydroxyapatites,” Biomaterials, 23, 751 (2002)
³Lee D D, Rey C, and Aiolova M. Synthesis of reactive amorphous calcium phosphates. U.S. Pat. No. 5,676,976, 1997

The reported literature values range from 8.0-9.6 ppm^{4,5} for calcium ion concentration in solution at equilibrium, depending on the substitution type and their amounts present.

The results show similar dissolution rates of these calcium phosphate materials in this in vitro system. The solubility of hydroxyapatite (HA) has been extensively investigated due to its relevance in bone tissue and its continued use in orthopedic devices. Synthetic apatites having similar mineralogic and chemical compositions can be expected to have similar rates of remodeling in vivo. Synthetic apatites with similar compositional and mineralogic characteristics as bone mineral have been shown to be remodeled via normal cellular activity.⁴ Ionic substitution disrupts the crystal structure and alters the dissolution properties of synthetic and biologic apatites. Some ionic substitutions such as carbonate have been shown to increase the solubility of apatites, while fluoride substitution into the apatite lattice decreases solubility.⁵ ⁴ E. P. Frankenberg, S. A. Goldstein, T. W. Bauer, S. A. Harris, and R. D. Poser, “Biomechanical and histological evaluation of a calcium phosphate cement,” Journal of Bone and Joint Surgery, 80, 1112 (1998)⁵ LeGeros R. Z. Biological and Synthetic Apatites. In Hydroxyapatite and related Materials. Brown and Constantz editors. 1994, CRC Press.

The calcium ion concentrations for both Skaffold NMX and predicate device (Callas®) are very similar and also match closely with those reported in literature for hydroxyapatite (8.0 ppm reported by Fulmer et al., and 9.6 ppm by Lee, et al.). These values differ depending on the substitution in the crystal lattice and their amounts present. Based on their similar in vitro solubility and dissolution rates at physiologic temperature and pH; Skaffold NMX and Callos® are anticipated to have similar in vivo remodeling rates.

### 18.3. Working Time

### Procedure

The working time of Skaffold NMX at 37° C. was determined using a variation of the setting test. The following addendum to the procedure provides details regarding test times and loads.

Testing was performed at 37° C. with mechanical testing machine using a modified high-load indenter with the crosshead travel speed set to 15.2 mm/min and a maximum load of 5000 N. No spring load average was calculated or used since the high load indenter test fixture does not use a spring. The prepared cement was then filled into the working time containers and placed in PBS bath maintained at 37° C. One sample was removed from the PBS at each test time point for testing. The sample was preloaded to 0.5N prior to the initiation of each test. The cement was tested at 6, 9, 12, 15 and 18 minutes post immersion into 37° C. PBS bath. After preloading, the high load indenter was allowed to travel 0.125 cm into each sample and the final load was recorded. The indentation load vs. time was plotted and an exponential curve fit on the plot generated. The working time is expressed at the time at which the first evidence of indentation load >27 Newtons is recorded. This study was performed in triplicates.

### Results

Three samples of Skaffold NMX were tested for the initiation of set. The individual data points, averages and standard deviations for all samples are tabulated in Table 7. The data yielded initiation of set times of 15 minutes to reach a recordable load greater than 27 Newtons when curing temperature was 37° C. This means that the maximum amount of working time allowed before the material begins to harden in vivo is 15 minutes following implantation. This provides ample opportunity for surgical adjustment if needed.

TABLE 7
Working time test for Skaffold NMX after immersing
in PBS at 37° C., values expressed in Newtons (N)

Time

| | 6 | 9 | 72 | 75 | 18 |
|---|---|---|---|---|---|
| Sample | minutes | minutes | minutes | minutes | minutes |
| 1 | 22 | 33 | 27 | 24 | 36 |
| 2 | 27 | 17 | 23 | 36 | 32 |
| 3 | 27 | 25 | 19 | 28 | 37 |
| Average | 25.33 | 25.00 | 23.00 | 29.33 | 35.00 |

### 18.4. Setting Time

The purpose of this test is to examine the setting properties of Skaffold NMX to ensure that the material sets sufficiently hard in vitro in a clinically relevant time frame under physiologic conditions of pH temperature.

### Procedure

This test is a modification of the standard setting test described in ASTM C403/C403M-99 in which the load required to drive needles a prescribed distance into concrete or a similar setting material is measured. The modification involves a needle with a tip configuration similar to that used in ASTM C266-99. The needle is pushed 0.125 mm into the sample cured under physiologic conditions (37° C. and 100% RH). An indentation load in excess of 3.5 MPa (135 Newton) has been determined as the time of initial setting according to the standard of ASTM C403/C403M-99. The indentation test is a sensitive measure of early solidification of Skaffold NMX.

A modified high load indentor (7 mm in diameter) was attached to Instron material testing machine with a maximum load of 5000 N. No spring load average was calculated or used in later calculations (the high load indentor test fixture does not use a spring).

Setting test containers were filled with Skaffold NMX, and placed in phosphate buffered saline (PBS) maintained at 37° C. Each sample was placed under the indenter carefully so that the center of the indenter is aligned with the center of the sample. Indenter was lowered so that the tip of the indentor was barely touching the surface of the sample. Test times for each sample were 0.5, 1, 2, 3 and 4 hours after the immersion into the PBS bath. The high load indenter was allowed to travel 0.125 cm into each sample with a speed of 15.2 mm/min, and the final load and test time were recorded.

Six (6) samples were measured for each time period. The mean loads and standard deviation were calculated for each test time. The indentation load vs. time was plotted, and an exponential curve fit on the plot was generated showing the equation and correlation coefficient.

### Results

Skaffold NMX reaches adequate strength within 1.5 hours after immersion in physiological conditions of temperature and pH as shown in FIG. 6. Interpolation of the curves created from the data yielded set time of ˜1.5 hours to reach a load greater than 3.5 MPa (135 Newton). The test outlined above was designed to assess the strength (setting) of the material over time in an in vivo simulated (i.e., in PBS at 37° C.) environment. All samples tested resulted in set time of 1.5 hours.

### 18.5. Dimensional Stability

### Procedure

Skaffold NMX was filled into the molds (Internal Diameter=0.5 inches and Height=0.3 inches) and placed in PBS at 37° C. allowing them to harden for 1 hour. After 1 hour, the heights of the samples in the respective mold were measured and recorded. The internal diameter remained constant for all samples. Specimens were then returned to the PBS bath and allowed to cure for a total of 24 hours at 37° C. and pH=7.4. Height measurements were then repeated and recorded. A total of 6 samples were tested for both devices, and for each sample, a total of 3 readings were recorded for height.

### Results

Table 8 shows the recorded height measurements for Skaffold NMX at 1 hour and 24 hours. The data shows that Skaffold NMX hardened at 1 hour in a contained volume exhibited no change in physical shape at 24 hours. Therefore, no change in physical shape would be expected following implantation in vivo.

TABLE 8
Dimensional stability data for Skaffold NMX after initial (1 hour) and
final soaking (24 hours) in PBS maintained at 37° C.

| | 1 hour | | | 24 hours | | | Change |
|---|---|---|---|---|---|---|---|
| Sample # | 1 | 2 | 3 | 1 | 2 | 3 | (inches) |
| 1 | 0.344 | 0.340 | 0.343 | 0.341 | 0.343 | 0.342 | 0.000 |
| 2 | 0.342 | 0.342 | 0.345 | 0.347 | 0.343 | 0.344 | −0.002 |
| 3 | 0.347 | 0.349 | 0.345 | 0.348 | 0.349 | 0.345 | 0.000 |
| 4 | 0.318 | 0.321 | 0.320 | 0.321 | 0.322 | 0.319 | −0.001 |
| 5 | 0.318 | 0.322 | 0.323 | 0.321 | 0.319 | 0.321 | 0.001 |
| 6 | 0.319 | 0.323 | 0.318 | 0.320 | 0.319 | 0.319 | 0.001 |

### 18.6. Setting Reaction Temperature

### Testing Compared to Predicate Device

### Procedure

Skaffold NMX was used as is and Callos was mixed according to the instructions for use. 10 grams for both cements was placed at the bottom of a 100 ml beaker containing 37° C. phosphate buffered saline (PBS). The centrifuge tube was placed in a 37° C. water bath. Thermocouples were placed at three locations during the course of each experiment:

### 1) Core

2) Surface (˜5 mm deep)
3) PBS bath

Temperature readings were recorded at 2 minute intervals for 24 Hours following introduction of the sample to the water bath. A total of three (3) samples were tested for both devices, subject and predicate.

### Results

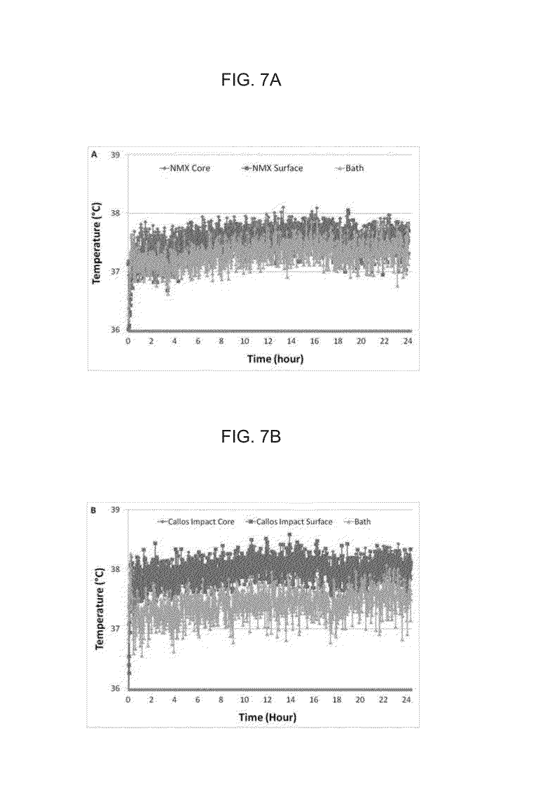

The results indicated that both Skaffold NMX (FIG. 7a) and Callos® (FIG. 7b) cure via an isothermic reaction (37° C.). These data indicate that the setting reaction of Skaffold NMX does not significantly change the temperature of fluids in the immediate vicinity of the setting material. Minimization of temperature fluctuation is expected to ensure tissue compatibility. In conclusion, the results demonstrate that Skaffold NMX remains within the physiologic temperature range and would be expected to show no adverse biologic consequence.

### 18.7. Chemical Analysis

The following tests were performed to chemically analyze the Skaffold NMX. All tests were performed on predicate, Callos® (K051123) to establish substantial equivalence:
1. Chemistry—Fourier Transform Infrared Spectroscopy and X-ray Diffraction
2. Crystallinity—X-ray Diffraction
3. Physical Form—Scanning Electron Microscope
4. Porosity—Mercury Intrusion Porosimetry

18.7.1. Chemistry—Testing Compared to Predicate Device

18.7.1.1. Fourier Transformed Infrared Spectroscopy

### Procedure

Skaffold NMX was used as is and Callos® was mixed according to the instructions for use. Both cements were filled into the molds (Internal Diameter=0.5 inches and Height=0.3 inches) and cured in PBS at 37° C. for 72 hours. After the first 24 hours, the cements were removed from the molds and placed back in the same PBS solution at previous conditions. Afterwards, the cured cements were dried at 37° C. for 72 hours. The dried cements (Skaffold NMX and Callos®) were ground in an agate mortar and sample pellets were made by mixing 1 mg of the dried cement powders: (a.) Skaffold NMX and (b.) Callos with 300 mg of dried spectroscopic grade KBr and compressed in a vacuum die under a pressure of 50,000 psi. The pellets were run on a FTIR machine (Nicolet iS10, Thermo-Nicolet, Woburn, Mass.) at 64 scans at a typical resolution of 4 cm⁻¹.

### Results

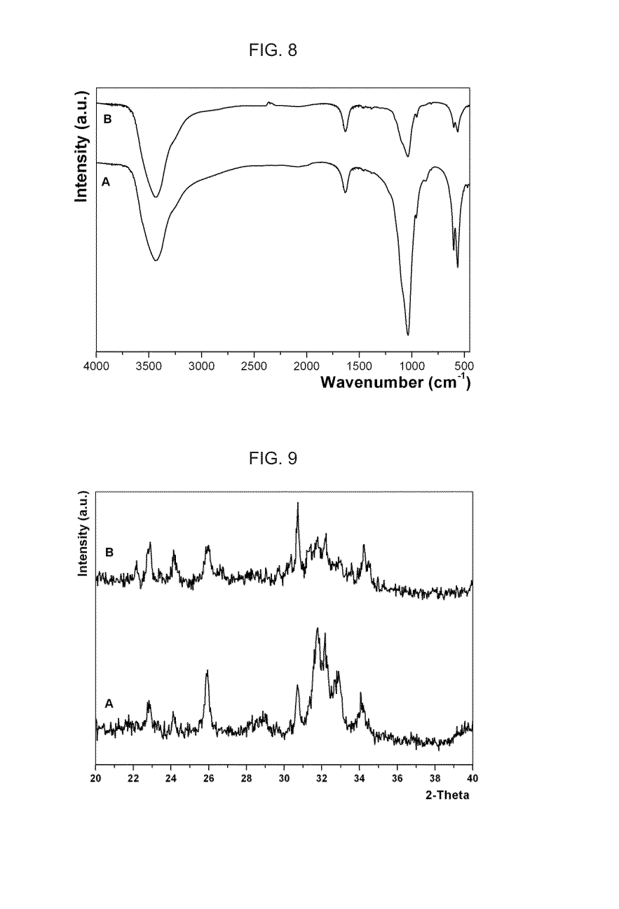

The comparison of FTIR patterns between Skaffold NMX and the predicate device (Callos®) is shown in FIG. 8. The FTIR patterns for both materials, Callos (trace A) and Skaffold NMX (trace B), match with each other. Both patterns show substantially pure hydroxyapatite with no other phases or compounds detected.

18.7.1.2. X-ray Diffraction

### Procedure

Skaffold NMX was used as is and Callos® was mixed according to the instructions for use. Both cements were filled into the molds (Internal Diameter=0.5 inches and Height=0.3 inches) and cured in PBS at 37° C. for 72 hours. After the first 24 hours, the cements were removed from the molds and placed back in the same PBS solution at previous conditions. Afterwards, the cured cements were dried at 37° C. for 72 hours. The dried cements (Skaffold NMX and Callos®) were ground in an agate mortar and samples were prepared for XRD powder diffraction. An Ultima IV powder X-ray diffractometer (Rikagu USA, The Woodlands, Tex.), operated at 40 kV and 44 mA, equipped with a Copper (Cu) tube was utilized to collect X-ray Diffraction patterns for these powder samples. X-ray diffraction data was collected for 2θ values of 20° to 40° with a step size of 0.02° 2θ and a preset time of 1 second at each step. The collected XRD patterns were then compared to the Powder Diffraction File 09-0432 as suggested by ASTM standard ASTM F1185.

### Results

FIG. 9 shows x-ray diffraction patterns for Callos® (trace A) and Skaffold NMX (trace B) following in vitro incubation for 72 hours. This diffraction pattern shows predominantly hydroxyapatite of low crystalline order with evidence of small amounts of crystalline starting reactant alpha-tricalcium phosphate. (ICDD #'s 9-432 and 9-0348, respectively). Both the subject device and predicate device matched to each other, with no other crystalline or amorphous phases present in either Skaffold NMX or Callos®.

18.7.2. Crystallinity—Testing Compared to Predicate Device

XRD was utilized to compare the crystallinity of Skaffold NMX with the predicate device. The results show that both materials consist of predominantly low crystalline hydroxyapatite with evidence of small amounts of crystalline starting reactant alpha-tricalcium phosphate. The results are demonstrated above in FIG. 9.

18.7.3. Physical Form—Testing Compared to Predicate Device

SKaffold NMX is available as a pre-mixed putty/paste intended to set in vivo and a Callos® (predicate device) kit consists of a powder and liquid components that need to be mixed to form a putty/paste that is intended to set in vivo. Scanning electron microscopy was used to compare the fully cured Skaffold NMX and predicate device.

### Procedure

Skaffold NMX was used as is and Callos was mixed according to the instructions for use. Both cements were filled into the molds (Internal Diameter=0.5 inches and Height=0.3 inches) and cured in PBS at 37° C. for 72 hours. After the first 24 hours, the cements were removed from the molds and placed back in the same PBS solution at previous conditions. Afterwards, the cured cements were dried at 37° C. for 72 hours and after drying cracked into two pieces. Surface morphology of the cracked surfaces of sputter-coated (w/Pt) Skaffold NMX and predicate device were evaluated with a scanning electron microscope (FE-SEM; FEI Quanta 650). The SEM was used in SE mode with an acceleration voltage of 10 kV.

### Results

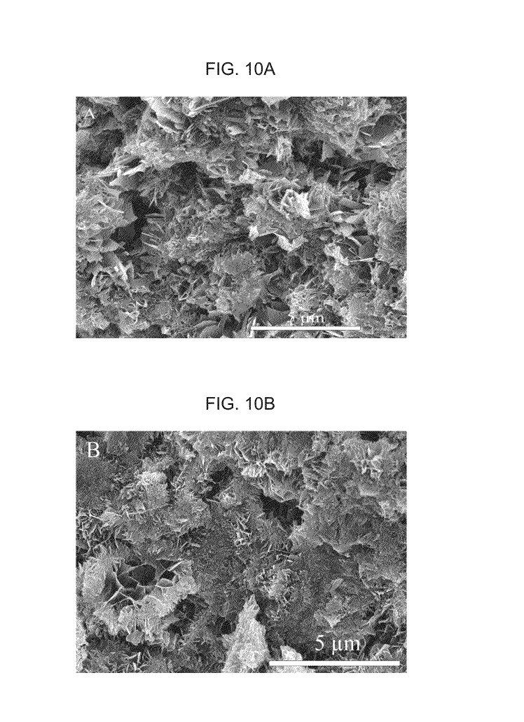

The SEM micrographs of cured and dried Skaffold NMX (FIG. 10A) showed that inter-mingling, inter-locking, nano-sized crystals of hydroxyapatite. Callos® after curing and dried (FIG. 10B) showed similar structure to that of Skaffold NMX with inter-mingling, inter-locking, nano-sized crystals of hydroxyapatite.

18.7.4. Porosity—Testing Compared to Predicate Device

### Procedure

Skaffold NMX was used as is and Callos® was mixed according to the instructions for use. The prepared cements were placed in molds—6 mm in diameter and 12 mm in height and then placed in PBS solution at 37° C. for 72 hours. After the first 24 hours, the cements were removed from the molds and placed back in the same PBS solution at previous conditions. After full curing, cements were dried at 37° C. for 24 hours and analyzed using mercury intrusion porosimetry. Mercury intrusion porosimetry was performed that compared the total porous volume, pore diameter and interconnectedness of Skaffold NMX with predicate device.

### Results

Table 9 shows the porosity data of Skaffold NMX and predicate device. The interconnected porosity for both devices, subject and predicate, was similar with Skaffold NMX being 41.1% and predicate device being 39.9%.

TABLE 9
Mercury Intrusion Porosimetry comparisons
between Skaffold NMX and predicate device

| | | |
|---|---|---|
| | | Predicate Device - |
| Intrusion Data | Skaffold NMX | Callos (K051123) |

| | | | | |
|---|---|---|---|---|
| Sample Mass | 0.4988 | g | 0.4987 | g |
| Sample Volume | 0.35 | ml | 0.36 | ml |
| Bulk Density | 1.43 | g/ml | 1.38 | g/ml |

| | | |
|---|---|---|
| Interconnected | 41.1% | 39.9% |
| Porosity | | |

| | | | | |
|---|---|---|---|---|
| Pore Diameter | 0.04 | microns | 0.03 | microns |

| | | |
|---|---|---|
| Total Intrusion | 0.29 ml pores | 0.29 ml pores |
| Volume (Total | per gram | per gram |
| Porous Volume) | Skaffold NMX | predicate device |

### CONCLUSION

The critical specifications of Skaffold NMX were compared to the predicate device.

These analyses consisted of:
Chemistry
Crystallinity
Physical form
Porosity
Dissolution/solubility
pH
Working time
Setting time
Dimensional stability
Setting reaction temperature

The results indicated that the device characteristics for Skaffold NMX were the same as those of the predicate device. Therefore, Skaffold NMX is substantially equivalent to the predicate device, Callos® calcium phosphate cement.

Notwithstanding the appended clauses, the disclosure set forth herein is also defined by the following clauses:

1. An implantable malleable calcium phosphate composition, the composition comprising:

(a) calcium and phosphate reactants that, upon combination with an aqueous medium, react to produce a calcium phosphate mineral that is different from the calcium and phosphate reactants; and

(b) a non-aqueous liquid carrier;

wherein upon contact with an aqueous medium, the calcium and phosphate reactants react with each other to produce a physiologically acceptable product comprising the calcium phosphate mineral.

2. The composition according to Clause 1, wherein the non-aqueous liquid carrier comprises a polyol.
3. The composition according to Clause 2, wherein the polyol comprises a polyalkylene oxide.
4. The composition according to Clause 3, wherein the polyalkylene oxide comprises a polyethylene glycol.
5. The composition according to Clause 4, wherein the polyethylene glycol is a low molecular weight polyethylene glycol.
6. The composition according to Clause 5, wherein the low molecular weight polyethylene glycol (PEG) has a molecular weight of 200 to 1,000.
7. The composition according to any of Clauses 1 to 6, wherein the calcium and phosphate reactants comprise a first particulate calcium phosphate reactant having a mean particle size of less than 8 μm and narrow particle size distribution.
8. The composition according to Clause 7, wherein the first particulate calcium phosphate reactant is a tricalcium phosphate.
9. The composition according to Clauses 7 or 8, wherein the composition further comprises a second calcium phosphate reactant having a mean particle size of 10 μm or greater.
10. The composition according to any of Clauses 1 to 9, wherein the composition further comprises a monovalent cation dihydrogen phosphate salt.
11. The composition according to any of Clauses 1 to 10, wherein the composition is a paste.
12. The composition according to any of Clauses 1 to 10, wherein the composition is a putty.
13. The composition according to any of Clauses 1 to 12, wherein the product has a compressive strength of 20 MPa or greater.
14. The composition according to Clause 13, wherein the product has a compressive strength of 50 MPa or greater.
15. The composition according to any of Clauses 1 to 14, wherein the product has a tensile strength of 0.5 to 10 MPa.
16. A method comprising:

applying an amount of a malleable calcium phosphate composition to a physiological site; and

allowing the applied composition to set into a solid product at the physiological site;

wherein the malleable calcium phosphate composition comprises:

(a) calcium and phosphate reactants that, upon combination with an aqueous medium, react to produce a calcium phosphate mineral that is different from the calcium and phosphate reactants; and

(b) a non-aqueous liquid carrier.

17. The method according to Clause 16, wherein the physiological site is a hard tissue defect site.
18. A method of making an implantable, malleable calcium phosphate composition, the method comprising:

combining calcium and phosphate reactants with a non-aqueous liquid carrier in a manner sufficient to produce the composition, wherein the calcium and phosphate reactants, upon combination with an aqueous medium, react to produce a calcium phosphate mineral containing product that is different from the calcium and phosphate reactants.

19. The method according to Clause 18, wherein the non-aqueous liquid carrier comprises a polyethylene glycol (PEG) having a molecular weight of 200 to 1,000.
20. A kit comprising:

a sterile package; and

an amount of an implantable, malleable calcium phosphate composition present in the sterile package, wherein the implantable, malleable calcium phosphate composition comprises:

(a) calcium and phosphate reactants that, upon combination with an aqueous medium, react to produce a calcium phosphate mineral that is different from the calcium and phosphate reactants; and

(b) a non-aqueous liquid carrier.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An implantable malleable calcium phosphate composition, the composition comprising:
(a) calcium and phosphate reactants that, upon combination with an aqueous medium, react to produce a calcium phosphate mineral that is different from the calcium and phosphate reactants; and
(b) a non-aqueous liquid carrier;
wherein upon contact with an aqueous medium, the calcium and phosphate reactants react with each other to produce a physiologically acceptable product comprising the calcium phosphate mineral.

2. The composition according to claim 1, wherein the non-aqueous liquid carrier comprises a polyol.

3. The composition according to claim 2, wherein the polyol comprises a polyalkylene oxide.

4. The composition according to claim 3, wherein the polyalkylene oxide comprises a polyethylene glycol.

5. The composition according to claim 4, wherein the polyethylene glycol is a low molecular weight polyethylene glycol.

6. The composition according to claim 5, wherein the low molecular weight polyethylene glycol (PEG) has a molecular weight of 200 to 1,000.

7. The composition according to claim 1, wherein the calcium and phosphate reactants comprise a first particulate calcium phosphate reactant having a mean particle size of less than 8 μm and narrow particle size distribution.

8. The composition according to claim 7, wherein the first particulate calcium phosphate reactant is a tricalcium phosphate.

9. The composition according to claim 7, wherein the composition further comprises a second calcium phosphate reactant having a mean particle size of 10 μm or greater.

10. The composition according to claim 1, wherein the composition further comprises a monovalent cation dihydrogen phosphate salt.

11. The composition according to claim 1, wherein the composition is a paste.

12. The composition according to claim 1, wherein the composition is a putty.

13. The composition according to claim 1, wherein the product has a compressive strength of 20 MPa or greater.

14. The composition according to claim 13, wherein the product has a compressive strength of 50 MPa or greater.

15. The composition according to claim 1, wherein the product has a tensile strength of 0.5 to 10 MPa.

16. A method comprising:
applying an amount of a malleable calcium phosphate composition to a physiological site; and
allowing the applied composition to set into a solid product at the physiological site;
wherein the malleable calcium phosphate composition comprises:
(a) calcium and phosphate reactants that, upon combination with an aqueous medium, react to produce a calcium phosphate mineral that is different from the calcium and phosphate reactants; and
(b) a non-aqueous liquid carrier.

17. The method according to claim 16, wherein the physiological site is a hard tissue defect site.

18. A method of making an implantable, malleable calcium phosphate composition, the method comprising:
combining calcium and phosphate reactants with a non-aqueous liquid carrier in a manner sufficient to produce the composition, wherein the calcium and phosphate reactants, upon combination with an aqueous medium, react to produce a calcium phosphate mineral containing product that is different from the calcium and phosphate reactants.

19. The method according to claim 18, wherein the non-aqueous liquid carrier comprises a polyethylene glycol (PEG) having a molecular weight of 200 to 1,000.

20. A kit comprising:
a sterile package; and
an amount of an implantable, malleable calcium phosphate composition present in the sterile package, wherein the implantable, malleable calcium phosphate composition comprises:
(a) calcium and phosphate reactants that, upon combination with an aqueous medium, react to produce a calcium phosphate mineral that is different from the calcium and phosphate reactants; and
(b) a non-aqueous liquid carrier.
